(19) United States
(12) Patent Application Publication
(10) Pub. No.: US 2024/0026114 A1
(43) Pub. Date: Jan. 25, 2024

(54) Depolymerization of Polyesters with Nano-Dispersed Enzymes

(71) Applicant: The Regents of the University of California, Oakland, CA (US)

(72) Inventors: Ting Xu, Berkeley, CA (US); Christopher A. DelRe, Berkeley, CA (US)

(73) Assignee: The Regents of the University of California, Oakland, CA (US)

(21) Appl. No.: 18/473,252

(22) Filed: Sep. 24, 2023

Related U.S. Application Data
(63) Continuation of application No. PCT/US22/24171, filed on Apr. 10, 2022.
(60) Provisional application No. 63/173,507, filed on Apr. 12, 2021.

(51) Int. Cl. C08J 11/10; C12N 9/20; C12N 9/50

(52) U.S. Cl. CPC C08J 11/105; C12N 9/20; C12N 9/50; C08J 2367/04; C12Y 304/21064; C12Y 301/01003

(57) ABSTRACT

Systems and methods for programmable degradation of a plastic deploy a plastic comprising a nanoscopic dispersion of enzymes and configured to exploit enzyme active sites and enzyme-protectant interactions to provide processive depolymerization as the primary degradation pathway with expanded substrate selectivity to effect substantially complete depolymerization without substantial microplastics formation with partial polymer degradation.

This invention was made with government support under the DA Army Research Office, contract number W911NF-13-1-0232, and the Department of Energy, grant number DE-AC02-05-CH11231. The government has certain rights in the invention.

### INTRODUCTION

Successfully interfacing enzymes and biomachineries with polymers affords on-demand modification and/or programmable plastic degradation during manufacture, utilization, and disposal, but requires controlled biocatalysis in solid matrices with macromolecular substrates.¹⁻⁷ Embedded enzyme microparticles have sped up polyester degradation, but compromise host properties and unintentionally accelerate microplastics formation with partial polymer degradation.^{6,8,9}

### SUMMARY OF THE INVENTION

We disclose that by nanoscopically dispersing enzymes with deep active sites, semi-crystalline polyesters can be degraded primarily via chain-end mediated processive depolymerization with programmable latency and material integrity, akin to polyadenylation-induced mRNA decay.¹⁰ We also disclose how to realize the processivity with enzymes having surface-exposed active sites by engineering enzyme/protectant/polymer complexes. By example, polycaprolactone and poly(lactic acid) containing less than 2 wt. % enzymes are depolymerized in days with up to 98% polymer-to-small molecule conversion in standard soil composts or household tap water, completely eliminating current needs to separate and landfill their products in compost facilities. Furthermore, oxidases embedded in polyolefins retain activities. However, the hydrocarbon polymers do not closely associate with enzymes like their polyester counterparts and the reactive radicals generated cannot chemically modify the macromolecular host. The disclosed molecular guidances provide enzyme/polymer pairing and enzyme protectants' selection to modulate substrate selectivity and optimize biocatalytic pathways.

The invention provides systems and methods for depolymerization of polyesters with nano-dispersed enzymes.

In an aspect the invention provides a system for programmable degradation of a plastic, comprising a plastic comprising a nanoscopic dispersion of enzymes and configured to exploit enzyme active sites and enzyme-protectant interactions to provide processive depolymerization as the primary degradation pathway with expanded substrate selectivity to effect substantially complete depolymerization without substantial microplastics formation with partial polymer degradation.

In an aspect the invention provides a method of programmable degradation of a plastic, comprising providing a plastic comprising a nanoscopic dispersion of enzymes and configured to exploit enzyme active sites and enzyme-protectant interactions to provide processive depolymerization as the primary degradation pathway with expanded substrate selectivity to effect substantially complete depolymerization without substantial microplastics formation with partial polymer degradation.

In embodiments:
a) the enzymes are lipase and the substrate is poly(caprolactone) (PCL), the lipase surface provides affinity to the substrate, and the binding site has a relatively narrow deep entrance;
b) the enzyme substrate is poly(lactic acid) (PLA) and the enzyme is proteinase K, and the proK binding site is relatively shallow and exposed;
c) the system comprises random heteropolymer (RHP) configured to nanoscopically disperse the enzymes and/or modulate activity or stability of the enzymes (e.g. serve as enzyme protectants).
d) the enzyme surface, protectant and polymer substrate form complexes to sandwich substrate in between;
e) the plastic comprises a semi-crystalline polyester;
f) the nanoscopic dispersion comprises about 0.001 to 5 wt % or 0.01 to 1.5 wt %;
g) the enzymes comprise a hydrolase, e.g. a lipase and/or a proteinase;
h) the depolymerization occurs in water or compost;
i) the depolymerization occurs in less than 1, 2, 5, or 10 days, e.g. in water, or less than 30, 60 or 90 days, e.g. in compost;
j) the depolymerization occurs at a temperature of 10-60 C, or 30-60 C, or about 37-40 C, wherein lower temperature (e.g. 10-30 C) depolymerization is facilitated by adjusting material properties, such as reducing crystalline lamellae thickness;
k) the plastic comprises a polyester, wherein the enzymes comprise an active site matched with the polyester backbone;
l) the plastic comprises lipase in poly(caprolactone) (PCL);
m) the plastic comprises proteinase in poly(lactic acid) (PLA);
n) the enzymes comprise a processive enzyme having a deep (e.g. about 1-4 nm, or about 2 nm), narrow (e.g. about 2-6 Å, or about 4.5 Å at the base) hydrophobic cleft from its surface to the catalytic site to facilitate substrate polymer-chain sliding while preventing dissociation;
o) the system comprises nanoscopically dispersed enzymes with deep active sites, and semi-crystalline polyesters degraded primarily via chain-end mediated processive depolymerization with programmable catalytic latency and material integrity;
p) the system realizes the processivity with enzymes having surface-exposed active sites by engineering enzyme/protectant/polymer complexes; and/or
q) polycaprolactone and poly(lactic acid) containing less than 2 wt. % enzymes are depolymerized in days with up to 98% polymer-to-small molecule conversion in standard soil composts or household tap water, eliminating needs to separate and landfill their products in compost facilities.

The invention encompasses all combinations of the particular embodiments recited herein, as if each combination had been laboriously recited.

### BRIEF DESCRIPTION OF THE DRAWINGS

Main Figure Legends

FIGS. 1A-C. Biocatalysis with embedded enzyme for polymer degradation. (A) Schematic illustrating two degradation pathways: plastic surface erosion with random chain scission and chain-end binding-mediated processive depolymerization when enzymes are nanoscopically confined to co-localize with polymer chain ends in the amorphous domain. The enzyme protectants (RHPs) are used to mediate enzyme-polymer interactions for dispersion and are rendered as chains of multi-coloured beads. (B) The reaction kinetic changes where macromolecular substrate binding becomes the rate-limiting factor with confined enzymes. The variables shown represent rate constants of a polymer chain diffusion into (kin) and out of (kout) the enzyme active site, and the catalytic reaction rate constant (kr). The enzymatic degradation rate is reduced when kin is the rate-limiting factor (kin<<kr). (C) Additional factors that modulate biocatalysis in solid states, as well as enzymatic reactions towards programmable polymer degradation. Left, a surface-exposed active site can readily bind chain segments, whereas a deep, narrow binding site prefers chain ends. Middle, the enzyme protectants (RHPs) can stabilize an enzyme, block active site or complex with a surface-exposed binding site to implement processivity. Right, semi-crystalline polymer chain conformation affects degradation rate.

FIGS. 2A-F. Characterization and degradation of PCL-RHP-BC-lipase. (A) Fluorescence microscope image of a film with homogeneously distributed fluorescently labelled BC-lipase. (B) Overlaid with an polarized optical microscope image. (C) Transmission electron microscope (TEM) image showing incorporation of RHP-lipase within semi-crystalline spherulites. (D) Stress-strain curve of PCL before and after RHP-BC-lipase incorporation. The inset shows a PCL-RHP-BC-lipase dog-bone sample before (left) and after (right) a tensile test. (E) SAXS profile of PCL-RHP-BC-lipase sample with 0, 10, 25 wt % weight loss. The inset shows a cross-sectional scanning electron microscope (SEM) image from a sample with 50% weight loss. (F) Fluorescence microscope image of microplastic particles formed after PCL-RHP-BC-lipase degraded in 40° C. buffer. Green fluorescently labelled BC-lipase remained uniformly distributed in the PCL matrix. The embedded enzymes continued to degrade PCL to achieve >95% PCL-small molecule conversion in one day.

FIGS. 3A-E. Embedded BC-lipase depolymerizes polyesters via chain end-mediated processive degradation. (A) Remaining mass (closed blue circles) and percent crystallinity (open black circles) of PCL-RHP-BC-lipase samples as a function of degradation time in 37° C. buffer (error bars represent one standard deviation; n≥3 for remaining mass, n≥2 for crystallinity). (b) GPC of PCL samples after surface erosion and confined degradation by BC-lipase, including the remaining film and degraded by-product. (C) Mass spectra of PCL degraded by surface erosion or by confined BC-lipase, including the remaining film and degraded by-product. The x axis (m/z) shows mass divided by charge. (D) Nuclear magnetic resonance (NMR) spectra of degradation by-products of PCL-b-PLA diblock copolymer when blended with RHP/BC lipase. Both small-molecule by-products of PCL and PLA were seen in BC lipase-containing diblock matrices, whereas only PCL degradation was observed for PCL-PLA blend matrices. The x axis (δ) shows the chemical peak shift. (E) Surface representation of BC-lipase and CA-lipase, highlighting the hydrophobic (white) substrate binding domain and the polar (purple) patch across from the binding domain; catalytic serine residue is shown in green, whereas negative and positive residues are shown in red and blue, respectively. a.u., arbitrary units.

FIGS. 4A-E. Enzyme protectants (RHPs) associate with the embedded enzyme to retain activity during melt processing and thermal treatment to program degradation. (A) Melt-extruded PCL-RHP-BC-lipase filaments containing about 0.1 wt % lipase that degrades into small molecules with near-complete conversion within 36 h in 40° C. buffer. (B) Programming of PCL-RHP-BC-lipase degradation by thermal treatment. Polarized optical imaging confirms that only regions with a low crystallization temperature are degraded after 24 h in 37° C. buffer. (C) Programming of PCL-RHP-BC-lipase degradation by degradation temperature. The degradation rate of PCL-RHP-BC-lipase is substantially suppressed below the onset of the PCL melting temperature or in amorphous PCL melt. This ensures PCL integrity during storage and melt processing. (D) RHPs can modulate depolymerization in PCL-BC-lipase and PLA-protease K. The remaining mass of PCL-BC-lipase shown is after 1 day of immersion in buffer, after 7 days for PLA-protease K with 20:50 MMA:EHMA RHP composition, and after 1 month for PLA-protease K with 50:20 and 60:10 MMA:EHMA RHP composites (n≥3). (E) Enzyme-containing PCL (left) and PLA (right) readily break down in ASTM standard composts.

FIGS. 5A-C. Characterization of enzyme-embedded PCL. (A) DLS results for RHP and purified BC-lipase in toluene (the solvent used to cast PCL) with an average hydrodynamic diameter of 285 nm±35 nm (n=5) (the error indicates standard deviation). (B) DSC results for PCL and PCL-RHP-BC-lipase as-cast films. (C) SAXS curves of PCL and PCL-RHP-BC-lipase as-cast films.

FIG. 6. PCL-RHP-BC-lipase by-product analysis. Liquid chromatogram of the degradation by-products for degradation by confined and dissolved (surface erosion) BC-lipase.

FIGS. 7A-B. Degradation by confined CA-lipase with shallow active site. (A) GPC curve of the degradation of PCL-RHP-CA-lipase, showing a shift and broadening of the main peak, indicative of random chain scission. (B) Zoomed-in version of a illustrating the peak shift and broadening.

FIGS. 8A-B. Enzyme environment dictates biocatalytic reaction kinetics. (A) PCL degradation by BC-lipase dissolved in solution (surface), nanoscopically embedded in PCL with RHP, and embedded with Tween 80, a small-molecule surfactant, as microparticles (error bars represent one standard deviation; n≥3). (B) Hydrolysis of p-nitrophenyl butyrate, a small-molecule ester, by BC-lipase in solution or confined in PCL.

FIGS. 9A-C. Model interfacial-tension experiment to explain intermolecular interactions among enzyme, protectant and matrix. When all three components are initially mixed in toluene (A, left) and then a water interface is introduced (A, right), RHP-lipase complexes immediately interact with PCL at the interface, as shown by the fluorescence microscopy image taken ˜20 s after shaking the vial to produce an emulsion (B) and the long delay time in interfacial-tension reduction that is seen only for PCL-RHP-lipase (C).

FIGS. 10A-B. Characterization of semi-crystalline properties of melt-processed PCL-RHP-BC-lipase (49° C., blue; As-cast, black). (A) DSC curves of PCL-RHP-BC-lipase with different recrystallization conditions (the film with recrystallization temperature Tc=49° C. has a crystallinity of 41%±1.2% compared to 39%±1.8% for the as-cast film). Heat Flow, ˜0.6 to W/g v. Temp, 40-7049° C. The increase in melting temperature from −58° C. to −64° C. indicates a substantial thickening in crystalline lamellae for Tc=49° C. films, which was confirmed by SAXS. (B) SAXS profiles of as-cast and Tc=49° C. films of PCL-RHP-BC-lipase. Intensity a.u. v. q×10⁴ Å⁻¹. The increase at long periods (shift to lower q), combined with the negligible difference in the bulk percent crystallinity according to DSC data, confirms a thickening in crystalline lamellae after crystallizing at Tc=49° C.

FIG. 11. Confirming enzyme does not denature at high temperatures. Small-molecule ester hydrolysis by embedded BC-lipase as a function of temperature (red), overlaid with the PCL-RHP-BC-lipase degradation rate. The small-molecule activity remained high at 60° C. but was not quantified because the film shriveled owing to melting, and thus was much thicker than films at lower temperatures, making quantification incomparable to all other temperatures.

FIGS. 12-A-D. Quantifying segmental hydrophobicity of different RHPs. (A) Hydropathy plots for RHPs with 60:10 MMA:EHMA composition. (B) Hydropathy plots for RHPs with 50:20 MMA:EHMA composition. (C) Hydropathy plots for RHPs with 20:50 MMA:EHMA composition. (D) Average segmental HLB value for each RHP composition. Error bars indicate standard deviation, n≥3.

FIGS. 13-A-E. Characterizing embedded enzymes for more commercially relevant plastics. (A) Crystal structure of proteinase K with the same colour-coding scheme as that used for lipases in the main text (FIGS. 3A-E). (B) GPC curve of PLA-RHP-proteinase K (“ProK”) as cast and after depolymerizing in buffer; (C) Interfacial-tensiometry experiment results for a DCM-water interface with PLA, RHP and proteinase K in the DCM phase. (D) Photograph of ABTS small-molecule assay in malonate buffer after ˜10 min, demonstrating that laccase embedded in polystyrene (PS) retains the ability to oxidize a small molecule. Similar results were found for manganese peroxidase and for both enzymes embedded in polyethylene. (E) Interfacial-tensiometry experiment results for a toluene-water interface with PS, RHP and either laccase or manganese peroxidase (‘MnP’) in the toluene phase.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

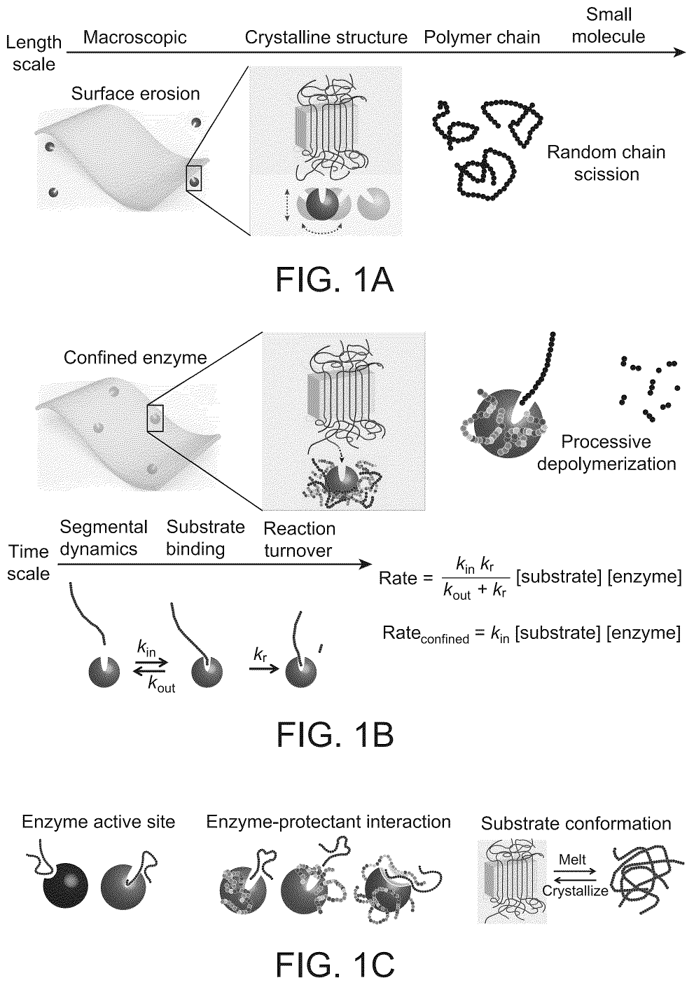

We envy nature's ability to program complex processes to achieve system-wide, long-term sustainability.¹¹⁻¹⁴ The key bottleneck is molecularly interfacing bio-elements with synthetic counterparts and, for enzyme-based plastic modification/degradation, how to manipulate biocatalysis with macromolecules being both the reaction substrates and host matrices.^{2,3,8,15} Enzymatic activity depends on the protein structure, substrate binding, and reactivity at the active site¹⁶⁻¹⁸. In semi-crystalline polymers, which represent the majority of plastics,¹³ substrate accessibility can be rate-limiting due to the reduced mobilities of the confined enzyme^{3,4,7} and polymer matrix¹⁹ (FIG. 1A and FIG. 1B). When polymers have chemically labile backbones, the enzyme can either randomly bind to and cleave a long chain or selectively bind to the chain end and catalyze depolymerization.^{20,21} Random chain scission has been the more prevalent pathway,^{6,14} but chain-end processive depolymerization is more desirable, since it directly and near completely converts a polymer to value-added monomers with near-complete degradation.^{16,22} Selective chain-end binding is challenging in solution biocatalysis,²³ but may become feasible when enzymes are nanoscopically confined to co-reside with the polymer chain ends. Solid state biocatalysis requires additional considerations that, if properly chosen, are beneficial (FIG. 1C). Thermodynamically, the polymer chain conformation contributes to the entropic gain, and thus, the global driving force of depolymerization. Kinetically, local polymer chain packing affects the segmental mobility and substrate binding to initiate and continue processive depolymerization.^{24,25} Protectants used to disperse the enzyme may compete for substrate binding and/or transiently modify the active sites, offering opportunities to regulate catalytic latency.^{5,26} Finally, the biocatalytic mechanism and types of targeted plastics must be considered.^{20,21,27} The degradation of condensation polymers, like polyesters, may only require substrate binding. Given their rapid market growth, understanding solid state enzymology can lead to immediate technological impact toward single use plastics.²⁸⁻³⁰ However, enzymatic modifications of chemically dormant molecules, such as hydrocarbons and/or polyolefins, require synchronization of multiple biocatalytic processes and are slow even under biologically optimized conditions.³¹ Without knowing how microbes modify and degrade polyolefins,^{15,21,32,33} understanding how embedded enzymes behave will guide protein engineering and the hybrid bio/abio catalysts design for plastic upcycling without generating secondary environmental contamination.

By nanoscopically confining enzymes in semi-crystalline polyesters and exploiting enzyme-active-site features and enzyme-protectant interactions, we show that processive depolymerization can be enabled as the primary degradation pathway with expanded substrate selectivity. Nanoscopic dispersion of a trace amount of enzyme, e.g., ˜0.02 wt. % lipase (<2 wt. % total additives) in poly(caprolactone), PCL, or ˜1.5 wt. % proteinase K (<5 wt. % total additives) in poly(lactic acid), PLA, leads to near-complete conversion to small molecules, eliminating microplastics in a few days using household tap water and standard soil composts. The programmable degradation overcomes their incompatibility with industrial compost operations, making them viable polyolefin substitutes.²⁸⁻³⁰ Analysis on the effects of polymer conformation and segmental cooperativity guide the thermal treatment of the polyester to spatially and temporally program degradation, while maintaining latency during processing and storage. The protectants are designed to regulate biocatalysis and stabilize enzymes during common plastic processing. Furthermore, with embedded oxidases such as laccase and manganese peroxidase, the enzymatically generated reactive radicals cannot oxidize the host polyolefins. There is a need to understand the biocatalytic cascades to design enzyme/host interactions and to enhance reactivity, diffusion, and lifetimes of reactive species without creating biohazards.

Biodegradable plastics PCL and PLA are market-ready alternates to many commodity plastics with increasing production and cost reduction.³⁴ However, they are indifferentiable in landfills.¹⁴ Typical residence times are not adequate to allow for full breakdown even in thermophilic digesters operating at 48-60° C., 28,29 resulting in operational challenges and a financial burden to minimize contamination in organic waste.³⁰ Burkholderia cepacia lipase (BC-lipase) and Candida Antarctica lipase (CA-lipase) were embedded in PCL and proteinase K was embedded in PLA given their known hydrolysis ability in solution.¹⁵ A previously developed four-monomer random heteropolymer (RHP) was added to nanoscopically disperse the enzymes.^{5,7} RHPs adjust the segmental conformations to mediate interactions between enzymes and local microenvironments.⁵ Extended Data Table 1 details the compositions of all blends.

Nano-Dispersed Lipase Accelerates PCL Degradation

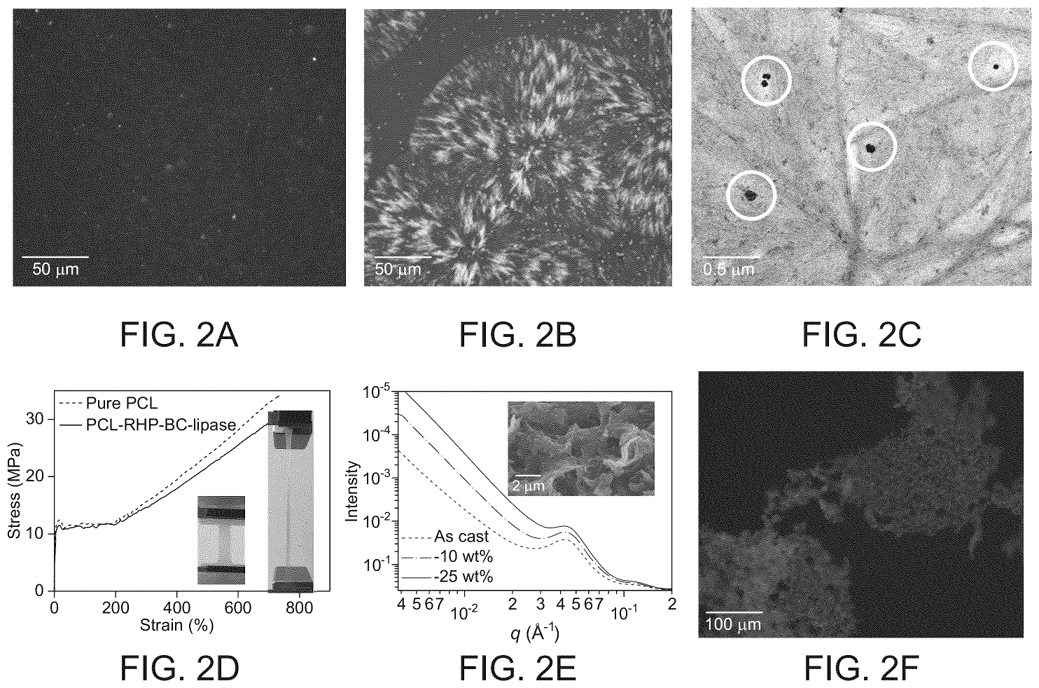

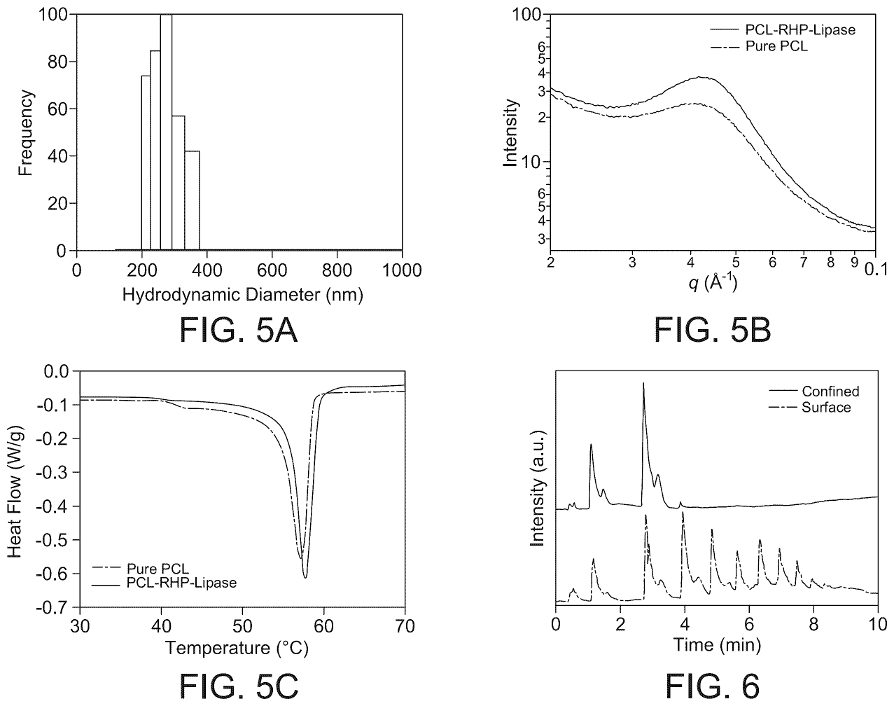

At 0.02-2 wt. % enzyme loading, RHP-lipase nanoclusters are uniformly distributed throughout (FIG. 2A, FIG. 5A) and incorporated within semi-crystalline spherulites (FIG. 2B). RHP-BC-lipase clusters, ˜50 nm to ˜500 nm in size, are located between bundles of PCL lamellae (FIG. 2C). A nanoscopic dispersion with minimal amounts of additives is key to retain host properties. Small angle x-ray scattering (SAXS) and differential scanning calorimetry (DSC) show similar PCL crystallization after lipase incorporation (FIG. 5B, 5C). With lipase-RHP loadings of up to 2 wt. %, there are less than 10% changes in the mechanical properties of PCL (FIG. 2D). The elastic modulus and tensile strength of PCL-RHP-BC-lipase are similar to those of low-density polyethylene (LDPE). PCL containing 0.02 wt. % BC-lipase degraded internally once immersed in a 40° C. buffer solution. Formation of nanoporous structure during internal degradation can be clearly seen in the cross-sectional scanning electron microscopy image and leads to increase in scattering intensity when the scattering vector q<0.04 Å⁻¹, due to enhanced contrast between the PCL and air (FIG. 2E). After disintegrated into microplastic particles (FIG. 2F), fluorescently labeled BC-lipase remained encapsulated and continued to degrade the microplastics to achieve ˜98% conversion within 24 hours.

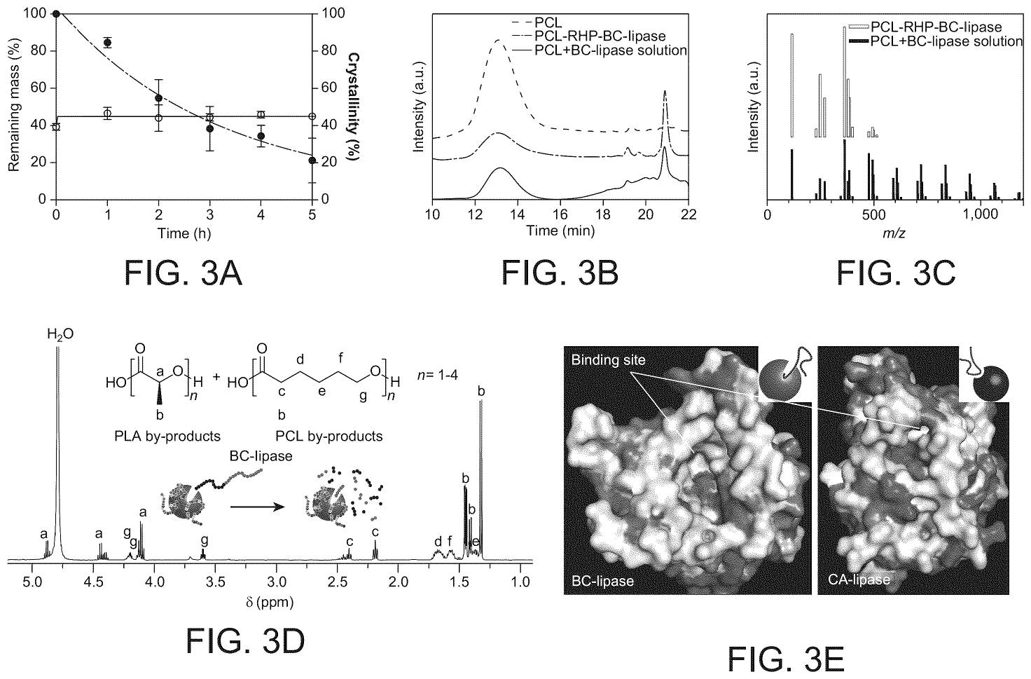

The overall PCL crystallinity in PCL-RHP-BC-lipase does not change when the degradation weight loss increased from 20% to 80% (FIG. 3A). Thus, the PCL segments in both the amorphous and crystalline phases degrade, as opposed to mainly the amorphous segments. This is consistent with the SAXS results in FIG. 2E where the peak position associated with lamellae periodicity does not change. The PCL molecular weight remains the same despite significant weight loss (FIG. 3B). The primary degradation by-products are repolymerizable small molecules, less than 500 Da in size (FIG. 3C, FIG. 6). Control experiments with PCL degradation via random chain scission show a wide range of high molecular weight oligomers. Thus, the degradation of PCL-RHP-BC-lipase should proceed via processive depolymerization.

Design Enzyme/Polymer Blends to Realize Processive Depolymerization

When BC-lipase nanoclusters are embedded in pure PLA or a PCL/PLA blend, no PLA hydrolysis is observed even though lipase catalyzes a broad range of hydrolysis reactions.³⁵ However, when the host matrix is a PCL-b-PLA diblock copolymer (40-b-20 kDa), both the PCL and PLA block depolymerize into small-molecules in a similar molar ratio as the parent copolymer (FIG. 3D). Thus, once a PCL chain end binds to the active site and is depolymerized by the BC-lipase, the PLA block can be shuttled to the active site and subsequently depolymerized. This is similar to polyadenylation-induced processive mRNA degradation,¹⁰ opening a useful route to expand substrate selection.

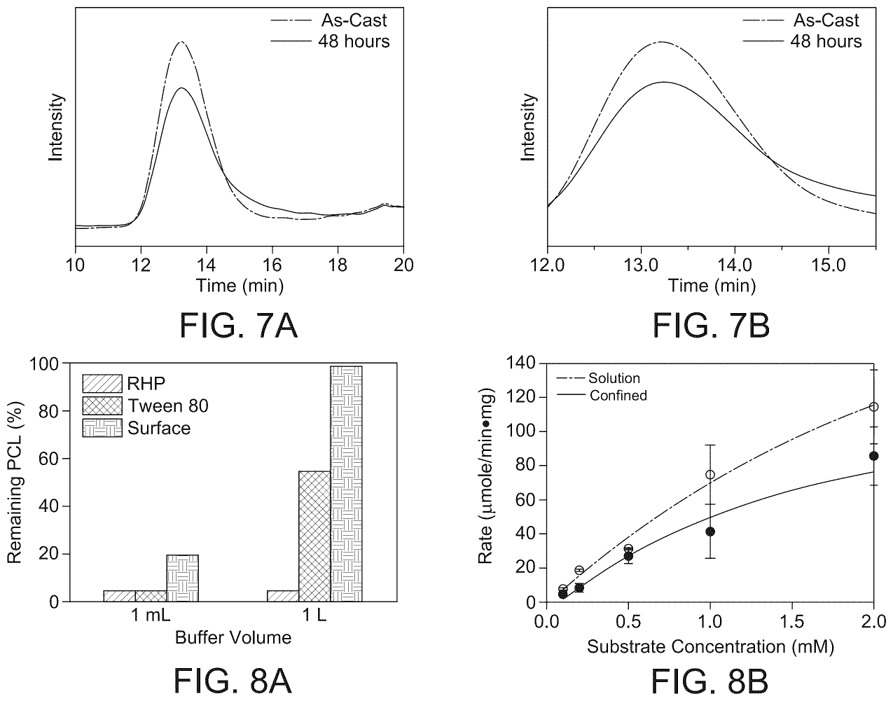

BC-lipase shares common traits with processive enzymes.^{23,24} It has a deep (up to 2 nm), narrow (4.5 Å at the base) hydrophobic cleft from its surface to the catalytic triad,¹⁷ which may facilitate substrate polymer chain sliding while preventing dissociation. Opposite to the hydrophobic binding patch are six polar residues, providing a potential driving force to pull the remaining chain forward after hydrolysis (FIG. 3E, left). Once the chain end is bound, the BC-lipase processively catalyzes the depolymerization without releasing it.²³ CA-lipase has a surface-exposed, shallow active site (˜1 nm from the surface) with no obvious residues that afford processivity (FIG. 3E, right). With random scission being the dominant pathway (FIGS. 7A-B), PCL-RHP-CA lipase degradation stopped after ˜12% mass loss and the bulk PCL crystallinity increased as degradation proceeded. Thus, the enzyme's surface chemistry and shape of the active site play important roles to modulate polymeric substrate binding toward preferential processive depolymerization.

Without nanoscopic confinement, BC-lipase degrades PCL via random chain scission in solution. When BC-lipase is embedded as micron-sized aggregates, the host degradation stops after ˜40% mass loss and leads to highly crystalline, long-lasting microplastics (FIG. 8A).^{6,8,9} Furthermore, PCL-RHP-BC-lipase undergoes negligible degradation at room temperature in buffer solution for >3 months, while BC-lipase in solution degrades ˜30% of pure PCL in 2 days. The hindered mobilities of the embedded enzyme and PCL segments limit initial substrate binding and depolymerization.

The turnover rate for embedded BC-lipase is ˜30 s⁻¹ for 0-3 hours and ˜12 s⁻¹ after 3 hours. The turnover rates of BC-lipase are ˜200 s⁻¹ in solution with small molecule substrate, ˜19 s⁻¹ in solution with a PCL film as substrate and ˜120 s⁻¹ in PCL-RHP-BC-lipase with a small molecule substrate (FIG. 8B). The embedded lipase shows a similar or higher apparent activity toward PCL than that in solution, where lipase has high rotational and translational freedom with higher substrate availability (i.e., polymer segments as opposed to chain ends). Thus, depolymerization kinetics are mainly governed by substrate binding for embedded enzymes and benefit significantly from chain end-mediated processive depolymerization pathway.

Therefore, to realize chain-end mediated processive depolymerization, the enzyme should be nanoscopically confined to co-reside with the polymer chain ends, exclude the middle segments from reaching the catalytic site, and have attractive interactions with the remaining chain end to slide the polymer chain without dissociation. With processive depolymerization, the host degrades with near-complete polymer-to-small molecule conversion, eventually eliminating highly crystalline microplastic particles. Kinetically, the apparent degradation rate benefits from substrate shuttling and catalytic latency can be regulated by thermal treatment and/or operation temperature.

Enzyme Protectants (RHPs) Modulate Enzyme Stability

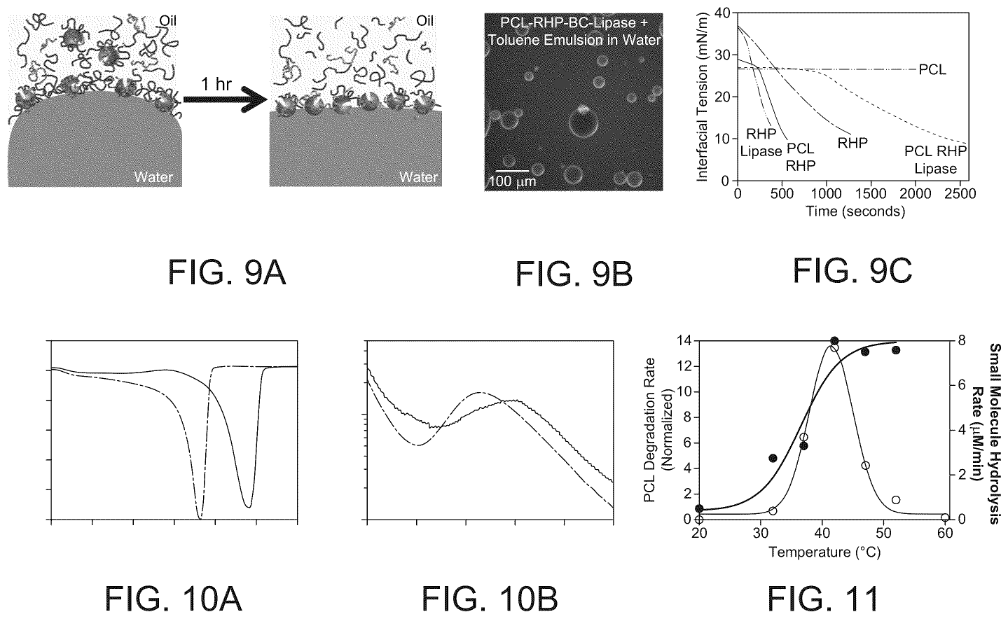

RHPs assist nanoscopic dispersion of enzymes and affect the local micro-environment, substrate accessibility, and possibly the degradation pathway. A model experiment at the solvent/water interface was designed where the interfacial tension is used to monitor molecular associations of the enzyme, RHP, and polymer (FIGS. 9A-B). Using pendant drop tensiometry, the toluene/water interfacial tension (y) decreases from 36 to 27 mN/m when PCL is in toluene, to ˜10 mN/m with lipase in water, and to less than 5 mN/m with only RHP in toluene. When all three components are in toluene, the interfacial tension is at 27 mN/m initially, remains unchanged for a period of time and then drops rapidly before plateauing at ˜7 mN/m and remains constant. Fluorescently labelled lipase immediately concentrates at the toluene/water interface (FIG. 9C). Taken together with the tensiometry data (FIG. 9D), RHP-lipase complexes concentrate at the toluene/water interface associated with PCL chains that wrap around the complexes. As lipase degrades PCL, the shorter chains desorb and expose the RHP-lipase complex, causing the reduction in tension. Thus, there is a coordinated interplay at the interface: PCL binds to the lipase and RHP facilitates the introduction of PCL into lipase, whereupon PCL degrades and leaves only the RHP/lipase complexes at the interface. Since the driving force for PCL to dissociate from lipase/RHP complex in dilute solution is higher than that in the melt, RHPs remain associated with lipase inside PCL.

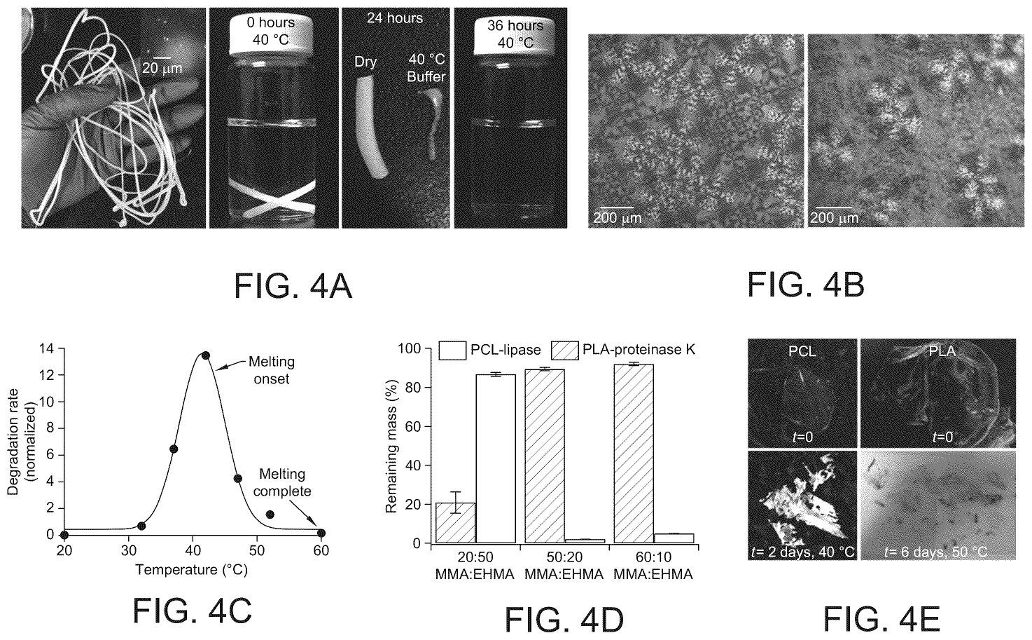

The RHPs modulate enzymes' micro-environment and provide entropic stabilization, enabling scalable processing of enzyme-embedded plastics using melt extrusion. PCL-RHP-BC-lipase containing ˜0.1 wt. % lipase was extruded at 85° C. to produce ˜1.5 mm diameter filament, which degraded completely over 36 hours in buffer by the same processive depolymerization mechanism (FIG. 4A).

Program Catalytic Latency

Polymer degradation can be programmed by thermal treatments. As the BC-lipase pulls the segments in the PCL stem spanning the crystalline lamellae, the competing force is governed by multiple pair-wise interactions between chains and degradation should not occur above a critical lamellae thickness. Indeed, PCL-RHP-BC-lipase films with thicker crystalline lamellae (crystallized at 49° C.) undergo negligible degradation over 3 months in 37° C. buffer, while films with thinner crystalline lamellae (crystallized at 20° C.) degrade over 95% in 24 hours (FIGS. 10A-B). This lamellae thickness dependence was exploited to spatially vary degradation within the same film (FIG. 4B). Control experiments using CA-lipase showed no dependence on thermal treatment or lamellae thickness, as expected with the random scission pathway.

Operation temperature is another handle to program degradation latency. There is a much lower conformational entropic penalty for a crystallized chain segment to bind to an enzyme than a completely amorphous chain.³⁶ The high entropic penalty for enzyme binding overtakes the effects of increased chain mobility, leading to large reductions in degradation rates at higher temperatures (>43° C.) (FIG. 4C) and eventually minimal PCL degradation in the melt state (>60° C.) despite the higher enzymatic activity against small molecule substrates (FIG. 11). These results counter the long-standing opinion that crystallinity slows enzymatic degradation of both synthetic^{18,20} and natural^{24,37} polymers, and enable exploitation of the chain-end mediated processive depolymerization to ensure catalytic latency and polymer integrity during melt processing and long-term storage.

Enzyme Protectants (RHPs) Modulate Catalytic Kinetics and Pathway

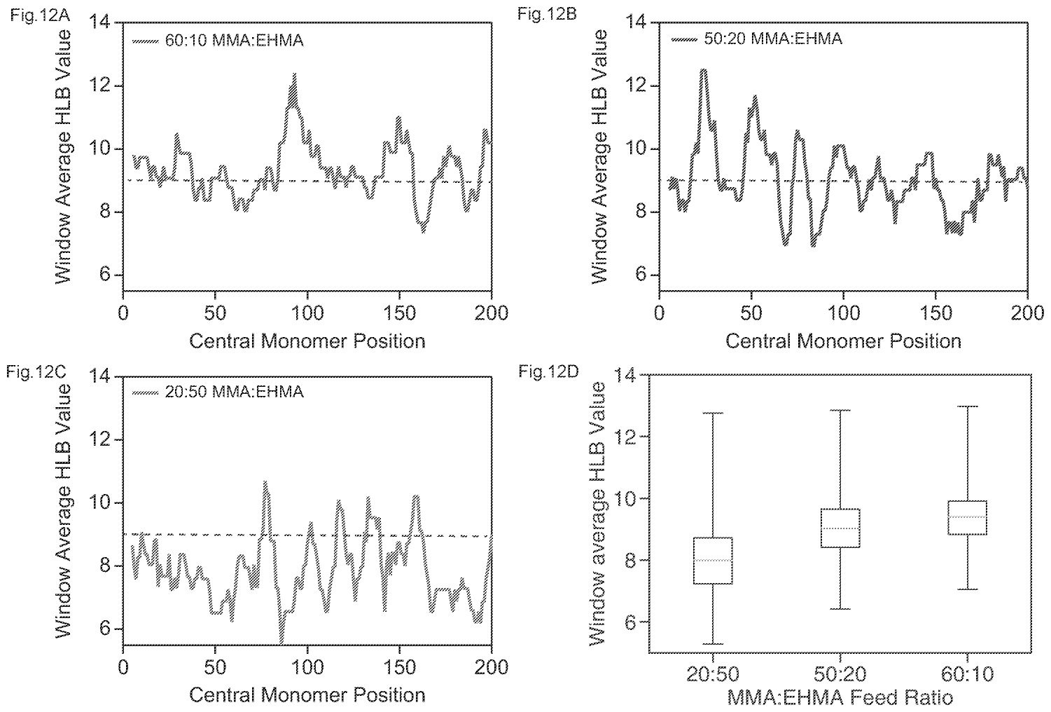

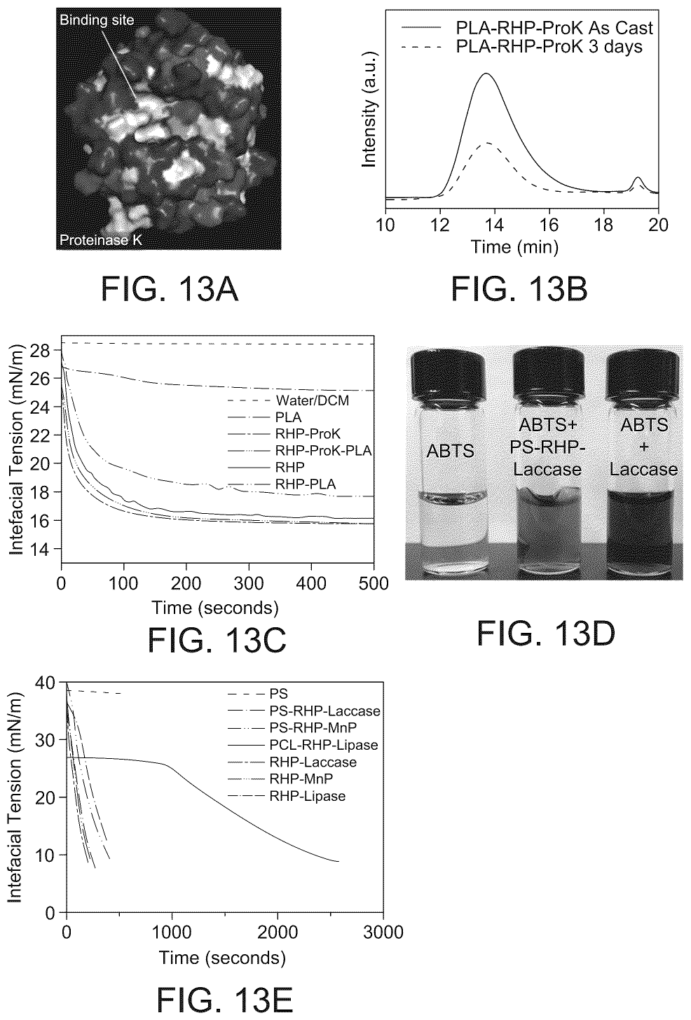

Proteinase K readily degrades PLA but the active site is highly surface-exposed, such that partial PLA degradation occurs with random chain scission, leaving highly crystalline microplastics behind. We hypothesize that modulating interactions between proteinase K binding site and RHPs may create an RHP-covered active site to achieve the characteristics of processive enzymes without protein engineering. We experimentally screened RHPs guided by the analysis of RHP segmental hydrophobicity³⁸ (FIGS. 12A-D) and the surface chemistry of proteinase K active site (FIG. 13A). The compositions of two hydrophilic monomers, oligo(ethylene glycol methyl ether methacrylate) (OEGMA) at 25% and sulfopropyl methacrylate potassium salt (SPMA) at 5%, are kept constant and the compositions of two hydrophobic monomers, methyl methacrylate (MMA) and ethyl hexyl methacrylate (EHMA) are varied. When the RHP with 20:50 MMA:EHMA composition is used, PLA depolymerized into small molecule byproducts readily without any observable change in the molecular weight or formation of intermediate molecular weight by-products (FIG. 4D, red, FIG. 13B). Tensiometry studies at the DCM/water interface confirmed proteinase K/RHP complexation and the PLA binding at the early stage of complexation (FIG. 13C). This suggests that the RHP binds to the enzyme surface to facilitate processivity by forming a hybrid “binding pocket” with proteinase K and shuttles in the PLA chains. However, when RHPs with compositions of 50:20 and 60:10 MMA:EHMA are used, minimal PLA depolymerization is observed with only ˜10% mass loss after 1 month in buffer despite high activity against a small molecule ester. Similarly, the RHP composition also affects the depolymerization rate of PCL (FIG. 4D, blue). Thus, besides being the enzyme protectants, RHPs can be designed to regulate substrate binding and active site availability, a useful handle to guide enzyme active-site engineering.³⁹ Experimentally, when 1.5 wt. % of proteinase K with 3 wt. % of RHPs are embedded, ˜80 wt. % PLA depolymerizes in 1 week in buffer at 37° C. Both enzyme-containing PCL and PLA show accelerated depolymerization in industrial soil composts (FIG. 4E), and films clearly disintegrate in a few days within the operating temperature range of industrial compost facilities (2 days at 40° C. for PCL and 6 days at 50° C. for PLA).

Hydrocarbon Substrate is Inaccessible to Embedded Oxidases

Besides synthetic catalysts, 22 biocatalysis of hydrocarbons is highly desirable due to its known efficiency, selectivity, and programmability.³¹ However, polyolefin degradation has mainly been reported using microbes, as opposed to enzymes.²¹ Polyolefin degradation is often initiated by side-chain modification, such as oxidation. To probe the bottlenecks, manganese peroxidase from white rot fungus and laccase from Trametes versicolor were embedded either in polyethylene or polystyrene with and without mediators (Tween 80 for manganese peroxidase and hydroxybenzotriazole for laccase). After two weeks in malonate buffer at 30° C. or 60° C., no changes are observed for any enzyme-polyolefin blends by infrared spectroscopy and gel permeation chromatography. For biosafety, these results are reassuring and expected with known longevity of plastic wastes. However, both enzymes remain highly active inside the plastics based on colorimetric assays, confirming formation of diffusive reactive radicals (FIG. 13D). Tensiometry studies confirm complexation between RHPs and both enzymes, but not between enzymes and polyolefins (FIG. 13E). The results suggest that the radicals generated cannot reach the polyolefin substrates, most likely due to limited diffusion, insufficient lifetime of reactive radicals, and the energy barrier to cross the interfacial layer between the enzyme and hydrocarbon chains.

Our technology enables fabrication of functional plastics with programmable life cycles compatible with plastic melt processing. Considering recent developments in synthetic biology and biodegradable plastic production, 14,34,39 modulating biocatalysis of embedded enzymes can provide molecular control over reaction pathway, kinetics, latency, and production of high value by-products.

### REFERENCES

1 Tokiwa, Y. & Suzuki, T. Hydrolysis of Polyesters by Lipases. Nature 270, 76-78, doi: 10.1038/270076a0 (1977).
2 Tournier, V. et al. An engineered PET depolymerase to break down and recycle plastic bottles. Nature 580, 216-219, doi:10.1038/s41586-020-2149-4 (2020).
3 Kuchler, A., Yoshimoto, M., Luginbuhl, S., Mavelli, F. & Walde, P. Enzymatic reactions in confined environments. Nat Nanotechnol 11, 409-420, doi:10.1038/nnano.2016.54 (2016).
4 Yang, Z. et al. Activity and Stability of Enzymes Incorporated into Acrylic Polymers. J Am Chem Soc 117, 4843-4850, doi: 10.1021/ja00122a014 (1995).
5 Panganiban, B. et al. Random heteropolymers preserve protein function in foreign environments. Science 359, 1239-1243, doi:10.1126/science.aao0335 (2018).
6 Ganesh, M., Dave, R. N., L'Amoreaux, W. & Gross, R. A. Embedded Enzymatic Biomaterial Degradation. Macromolecules 42, 6836-6839, doi:10.1021/ma901481h (2009).
7 DelRe, C. et al. Reusable Enzymatic Fiber Mats for Neurotoxin Remediation in Water. Acs Appl Mater Inter 10, 44216-44220, doi:10.1021/acsami.8b18484 (2018).
8 Khan, I., Nagarjuna, R., Dutta, J. R. & Ganesan, R. Enzyme-Embedded Degradation of Poly(epsilon-caprolactone) using Lipase-Derived from Probiotic Lactobacillus plantarum. Acs Omega 4, 2844-2852, doi:10.1021/acsomega.8b02642 (2019).
9 Huang, Q. Y., Hiyama, M., Kabe, T., Kimura, S. & Iwata, T. Enzymatic Self-Biodegradation of Poly(L-lactic acid) Films by Embedded Heat-Treated and Immobilized Proteinase K. Biomacromolecules 21, 3301-3307, doi:10.1021/acs.biomac.0c00759 (2020).
10 Xu, F. F. & Cohen, S. N. Rna Degradation in Escherichia-Coli Regulated by 3′ Adenylation and 5′ Phosphorylatian. Nature 374, 180-183, doi: 10.1038/374180a0 (1995).
11 Wei, R. et al. Possibilities and limitations of biotechnological plastic degradation and recycling. Nat Catal 3, 867-871, doi:10.1038/s41929-020-00521-w (2020).
12 Ivleva, N. P., Wiesheu, A. C. & Niessner, R. Microplastic in Aquatic Ecosystems. Angew Chem Int Edit 56, 1720-1739, doi:10.1002/anie.201606957 (2017).
13 Jambeck, J. R. et al. Plastic waste inputs from land into the ocean. Science 347, 768-771, doi:10.1126/science.1260352 (2015).
14 Haider, T. P., Volker, C., Kramm, J., Landfester, K. & Wurm, F. R. Plastics of the Future? The Impact of Biodegradable Polymers on the Environment and on Society. Angew Chem Int Edit 58, 50-62, doi:10.1002/a nie.201805766 (2019).
15 Roohi et al. Microbial Enzymatic Degradation of Biodegradable Plastics. Curr Pharm Biotechno 18, 429-440, doi:10.2174/1389201018666170523165742 (2017).
16 Breyer, W. A. & Matthews, B. W. Structure of Escherichia coli exonuclease I suggests how processivity is achieved. Nat Struct Biol 7, 1125-1128 (2000).
17 Pleiss, J., Fischer, M. & Schmid, R. D. Anatomy of lipase binding sites: the scissile fatty acid binding site. Chem Phys Lipids 93, 67-80, doi: 10.1016/S0009-3084(98)00030-9 (1998).
18 Li, S. M. & McCarthy, S. Influence of crystallinity and stereochemistry on the enzymatic degradation of poly(lactide)s. Macromolecules 32, 4454-4456, doi: (1999).
19 Flory, P. J. & Yoon, D. Y. Molecular Morphology in Semi-Crystalline Polymers. Nature 272, 226-229, doi: 10.1038/272226a0 (1978).
20 Tokiwa, Y., Calabia, B. P., Ugwu, C. U. & Aiba, S. Biodegradability of Plastics. Int J Mol Sci 10, 3722-3742, doi:10.3390/ijms10093722 (2009).
21 Ru, J. K., Huo, Y. X. & Yang, Y. Microbial Degradation and Valorization of Plastic Wastes. Front Microbiol 11, doi:ARTN 442 10.3389/fmicb.2020.00442 (2020).
22 Tennakoon, A. et al. Catalytic upcycling of high-density polyethylene via a processive mechanism. Nat Catal 3, 893-901, doi:10.1038/s41929-020-00519-4 (2020).
23 Horn, S. J. et al. Costs and benefits of processivity in enzymatic degradation of recalcitrant polysaccharides. P Natl Acad Sci USA 103, 18089-18094, doi:10.1073/pnas.0608909103 (2006).
24 Beckham, G. T. et al. Molecular-Level Origins of Biomass Recalcitrance: Decrystallization Free Energies for Four Common Cellulose Polymorphs. J Phys Chem B 115, 4118-4127, doi:10.1021/jp1106394 (2011).
25 Payne, C. M., Himmel, M. E., Crowley, M. F. & Beckham, G. T. Decrystallization of Oligosaccharides from the Cellulose I beta Surface with Molecular Simulation. J Phys Chem Lett 2, 1546-1550, doi:10.1021/jz2005122 (2011).
26 Klibanov, A. M. Improving enzymes by using them in organic solvents. Nature 409, 241-246, doi: 10.1038/35051719 (2001).
27 Christensen, P. R., Scheuermann, A. M., Loeffler, K. E. & Helms, B. A. Closed-loop recycling of plastics enabled by dynamic covalent diketoenamine bonds. Nat Chem 11, 442-448, doi:10.1038/s41557-019-0249-2 (2019).
28 Satchwel, A. J. et al. Accelerating the Deployment of Anaerobic Digestion to Meet Zero Waste Goals. Environ Sci Technol 52, 13663-13669, doi:10.1021/acs.est.8b04481 (2018).
29 Hobbs, S. R., Parameswaran, P., Astmann, B., Devkota, J. P. & Landis, A. E. Anaerobic Codigestion of Food Waste and Polylactic Acid: Effect of Pretreatment on Methane Yield and Solid Reduction. Adv Mater Sci Eng 2019, doi:Artn 4715904 10.1155/2019/4715904 (2019).
30 Nordahl, S. L. et al. Life-Cycle Greenhouse Gas Emissions and Human Health Trade-Offs of Organic Waste Management Strategies. Environ Sci Technol 54, 9200-9209, doi:10.1021/acs.est.0c00364 (2020).
31 Radi, R. Oxygen radicals, nitric oxide, and peroxynitrite: Redox pathways in molecular medicine. P Natl Acad Sci USA 115, 5839-5848, doi:10.1073/pnas.1804932115 (2018).
32 Iiyoshi, Y., Tsutsumi, Y. & Nishida, T. Polyethylene degradation by lignin-degrading fungi and malaganese peroxidase. J Wood Sci 44, 222-229, doi: 10.1007/Bf00521967 (1998).
33 Yang, J., Yang, Y., Wu, W. M., Zhao, J. & Jiang, L. Evidence of Polyethylene Biodegradation by Bacterial Strains from the Guts of Plastic-Eating Waxworms. Environ Sci Technol 48, 13776-13784, doi:10.1021/es504038a (2014).
34 Schneiderman, D. K. & Hillmyer, M. A. 50th Anniversary Perspective: There Is a Great Future in Sustainable Polymers. Macromolecules 50, 3733-3750, doi:10.1021/acs.macromol.7b00293 (2017).
35 Liu, L. J., Li, S. M., Garreau, H. & Vert, M. Selective enzymatic degradations of poly(L-lactide) and poly(epsilon-caprolactone) blend films. Biomacromolecules 1, 350-359, doi:10.1021/bm000046k (2000).
36 Varmanair, M., Pan, R. & Wunderlich, B. Heat-Capacities and Entropies of Linear, Aliphatic Polyesters. J Polym Sci Pol Phys 29, 1107-1115, doi: (1991).
37 Hall, M., Bansal, P., Lee, J. H., Realff, M. J. & Bommarius, A. S. Cellulose crystallinity—a key predictor of the enzymatic hydrolysis rate. Febs J 277, 1571-1582, doi:10.1111/j.1742-4658.2010.07585.x (2010).
38 Jiang, T. et al. Single-chain heteropolymers transport protons selectively and rapidly. Nature 577, 216-220, doi:10.1038/s41586-019-1881-0 (2020).
39 Arnold, F. H. Directed Evolution: Bringing New Chemistry to Life. Angew Chem Int Edit 57, 4143-4148, doi:10.1002/anie.201708408 (2018).

Section M1. Embedding Random Heteropolymer-Enzymes in Polyesters

Amano PS Lipase from Burkholderia cepacia (BC-lipase), Candida Antarctica Lipase B (CA-Lipase), and proteinase K from Tritirachium album were purchased from Sigma Aldrich. The BC-enzyme solution was purified following established procedure.⁴⁰ Proteinase K was purified by using a 10,000 g/mole molecular weight cutoff filter by spinning in a centrifuge at 6,000 rcf for 3 total cycles. The concentration of the purified lipase and proteinase K stock solution was determined using UV-vis absorbance at 280 nm. Detailed information for all samples is listed in Table 51.

The random heteropolymer (RHP) (70 KDa, PDI=1.55) was synthesized.⁵ The monomer molar composition used, unless otherwise specified, was 50% methyl methacrylate (MMA), 20% 2-ethylhexyl methacrylate (EHMA), 25% oligo(ethylene glycol methyl ether methacrylate) (OEGMA; Mn=500 g/mole), and 5% 3-sulfopropyl methacrylate potassium salt (SPMA). The RHP is referred as MMA:EHMA:OEGMA:SPMA=0.5:0.2:0.25:0.5. Two RHP variants were used to perform experiments described in FIG. 4E and the composition is MMA:EHMA:OEGMA:SPMA=0.6:0.1:0.25:0.05 and MMA:EHMA:OEGMA:SPMA=0.2:0.5:0.25:0.05, respectively.

RHP and enzymes were mixed in aqueous solution, flash-frozen in liquid nitrogen, and lyophilized overnight. The dried RHP-enzyme mixture was resuspended directly in the specified polymer solutions or melts. RHP was mixed with purified BC-lipase in a mass ratio of 80:1 (total polymer matrix mass=98.4%). For commercial BC-lipase and CA-lipase blends, the RHP to blend weight ratio was kept at 2:1 (total polymer matrix mass=95.5%). For proteinase K in PLA, a 2:1 RHP:enzyme ratio was used (total polymer matrix mass=95.5%).

PCL (80 KDa) and PLA (85-160 KDa) were purchased from Sigma Aldrich and used without further purification. To prepare solution-cast films, PCL (or PLA) was dissolved in toluene (or dichloromethane) at 4 wt. % concentration and stirred for at least 4 hours to ensure complete dissolution. The dried RHP-enzyme complexes were resuspended at room temperature directly in the polymer solution at the specified enzyme concentration. Mixtures were vortexed for ˜5 mins before being cast directly on a glass plate. PCL films were air dried and PLA films were dried under a glass dish to prevent rapid solvent evaporation given the volatility of dichloromethane.

To probe enzyme distribution, lipase was fluorescently labeled. NHS-Fluorescein (5/6-carboxyfluorescein succinimidyl ester) was used to label lipase and remove excess dye by following manufacturer's procedure. A U-MWBS3 mirror unit with 460-490 nm excitation wavelengths was used to take the fluorescence microscopy images. TEM images were taken on a JEOL 1200 microscope at 120 kV accelerating voltage. Vapor from a 0.5 wt. % ruthenium tetroxide solution was used to stain the RHP-lipase and the amorphous PCL domains.

Section M2. Characterization of as-Cast Plastics

Dynamic light scattering (DLS) was used to obtain the complex's particle size in toluene. Crystallinity and mechanical properties of enzyme-embedded polyesters were probed via differential scanning calorimetry (DSC) and tensile testing, respectively. For DSC, ˜5 mg PCL films were pressed into aluminum pans and heated from 25° C. to 70° C. at a 2° C./min scan rate. To quantify percent crystallinity, the sample's enthalpy of melting was normalized by 151.7 J/g, enthalpy of melting for 100% crystalline PCL.⁴¹ For uniaxial tensile tests, PCL solutions were cast directly in custom-designed Teflon molds with standard dog-bone shapes. For small angle x-ray scattering (SAXS) studies, ˜300 μm thick films were cast in Teflon beakers. Samples were vacuum dried after degradation for 16 hours prior to running SAXS at beamline 7.3.3 at the Advanced Light Source (ALS). X-rays with 1.24 Å wavelength and 2 s exposure times were used. The scattered X-ray intensity distribution was detected using a high-speed Pilatus 2M detector. Images were plotted as intensity (I) vs. q, where q=(4π/λ) sin(θ), λ is the wavelength of the incident X-ray beam, and 2θ is the scattering angle. The sector-average profiles of SAXS patterns were extracted using Igor Pro with the Nika package. The same SAXS method was used to analyze the nanoporous structure of samples at different time points of the degradation process, as shown in FIG. 2E. To obtain the cross-sectional SEM image shown in the inset to FIG. 2E, the degraded film was rinsed and fractured in liquid nitrogen. The film was then mounted on an SEM stub and sputter coated with platinum prior to imaging.

Section M3. Characterization of Enzyme-Embedded PCL Degradation

Degradation was carried out in sodium phosphate buffer (25 mM, pH 7.2) at temperature specified. Mass loss was determined by drying the remaining film and measuring mass on a balance. After 24 hours, mass loss was estimated by integrating gel permeation chromatography (GPC) peaks. The microplastic experiment shown in FIG. 2F was run with a ˜5 mg PCL-RHP-BC-lipase film (0.02 wt. % enzyme) in 3 mL of buffer at 40° C. The same experiment was run with fluorescently labeled enzyme.

At each timepoint from 0-5 hours, PCL-RHP-BC-lipase remaining films were dried and analyzed via DSC to determine crystallinity. To analyze degradation by-products, vials were lyophilized overnight before resuspending in the proper solvent for GPC or LCMS. GPC measurements were run using a total concentration of 2 mg/mL of remaining film and by-product in THF. 20 uL of solution was injected into an Agilent PolyPore 7.5×300 mm column; GPC spectrum for BC-lipase in solution was normalized to the solvent front. Liquid chromatography-mass spectrometry (LC-MS) measurements were obtained by resuspending degradation supernatant in acetonitrile/water (67/33 vol %), using an Agilent InfinityLab EC-C18, 2.7 μm column. Control experiments for surface erosion were run with ˜0.15 mg/mL total BC-lipase blend concentration. The mass spectrum shown in FIG. 3C is a combination of the major peaks seen in the chromatogram (FIG. 6). The by-products were repolymerized as proof-of-concept using a previously-reported method⁴² after recovering degraded PCL by-product from enzyme and buffer salts via phase extraction and filtration.

Section M4. Enzyme Active Site Affects Degradation by Confined Enzymes

RHP-BC-lipase was embedded in a PCL-b-PLA deblock copolymer blended with pure PLA for the testing because the diblock on its own was too brittle to form a freestanding film after drying. The film was cast from a solution of 9 wt. % PCL-b-PLA (purchased from Polymer Source)+4 wt. % pure PLA in dichloromethane. The film was allowed to degrade at 40° C. buffer for 24 hours, and the by-products were analyzed using NMR. Similar results were obtained for homemade PCL-b-PLA diblock copolymer without any blended pure PLA homopolymer (10k-b-8k based on NMR analysis).

Crystal structures of BC-lipase and CA-lipase are taken from entries SLIP and 1TCA in protein data bank, respectively. Analysis of proteinase K active site was carried out using entry 3PRK. Hydrophobic residues (gray) are defined as the following amino acids: alanine, glycine, valine, leucine, isoleucine, phenylalanine, methionine, and proline. Aspartic acid and glutamic acid are defined as negative residues (red), while lysine, arginine, and histidine are defined as positive residues (blue). The remaining residues are considered polar uncharged residues (purple). GPC on PCL-RHP-CA-lipase films (degraded in 37° C. buffer) was carried out following the same procedure as for BC-lipase-embedded films.

Section M5. Confinement Affects Degradation Pathway: Nanoscopic Vs. Microscopic Vs. Surface Erosion

Degradation was run in a 1 mL and 1 L container while shaking the container every few hours to demonstrate the effects of enzyme leaching and diffusion. PCL-RHP-BC-lipase degrades similarly in both volumes (≥95% degradation in 24 hours), consistent with internal degradation and limited enzyme leaching.

Pure PCL films were placed in 1 L buffer with an equivalent mass of total lipase as was present in the PCL-RHP-lipase films. Pure PCL films exhibited negligible degradation in 1 L buffer over a week, whereas pure PCL films in 1 mL buffer with the same enzyme mass lost ˜80% mass in 1 day. This buffer volume dependence is expected, because enzyme must diffuse to plastic surface in order to hydrolyze the plastic.

To simulate experiments detailed in previous literature for comparison, 6,8 Tween80 was mixed with purified lipase in a 1:1 mass ratio, lyophilized, and resuspended in PCL/toluene to cast films. In 1 L buffer, films with Tween80-embedded enzyme at the same enzyme loading as PCL-RHP-BC-lipase degraded by ˜40% in 1 day and then stopped degrading (monitored over 1 week), whereas in 1 mL buffer the small molecule-embedded film degraded similarly to RHP-embedded film (≥95% in 24 hours). This reliance on buffer volume suggests that small molecule surfactant-embedded enzyme experiments previously reported in literature exhibit significant leaching, and in large volumes this enzyme leaching prevents complete polymer degradation.

Section M6. Kinetic Analysis of BC-Lipase in Different Environments with Different Substrates

M6.1 Confined BC-lipase with PCL substrate: The slope of the degradation plot shown in FIG. 2A was used to estimate the degradation rate for confined lipase at 37° C. Two different slopes were obtained (0-3 hours and 3-5 hours) and the rate changed around 3 hours. The turnover rate was determined by dividing the number of PCL bonds broken per second by the total number of lipase molecule in the film, assuming an average trimer PCL by-product based on the LC-MS by-product analysis.

M6.2 Dissolved BC-lipase with PCL substrate: Pure PCL films (˜5 mg each) were placed in 1 mL buffer (37° C.) containing ˜1 lag of lipase to mimic concentrations from degradation experiments of confined lipase. The turnover rate provided in the text was determined by also assuming a trimer by-product, which may represent an upper bound since surface erosion can occur by random scission (larger oligomers generated per bond cleavage would serve to reduce the apparent turnover rate since more mass is lost per bond cleavage).

M6.3 Dissolved and confined BC-lipase with small molecule substrate: The same small molecule assay was used to quantify activity of dissolved and confined BC-lipase. 4-nitrophenyl butyrate was dissolved in buffer at each substrate concentration prior to running the assay to rule out interfacial effects of soluble lipase. Activity was quantified via UV-vis to monitor the absorbance over 10 mins of the hydrolyzed by-product at 410 nm. Extinction coefficient for by-product was estimated as 16,500 M⁻¹ cm⁻¹. PRISM software was used to fit the activity as a function of substrate concentration in order to obtain Vmax, the theoretical maximum reaction rate at saturated substrate concentration. Vmax was converted to a turnover rate by converting per-mass to per-lipase molecule. The same small molecule assay was used to quantify activity of confined lipase in PCL.

Section M7. Dynamic Interfacial Tension Experiments to Probe PCL-RHP-Lipase Interactions

Interfacial tension between a toluene and water phase was used to probe the blends. A MilliQ water droplet was dispensed by a 1 mL syringe through a 1.27 mm-diameter needle and immersed in toluene. The droplet shape was captured by a CCD camera every second and fitted by Young-Laplace equation to obtain interfacial tension. For each sample, the measurement was repeated three times and showed good consistency and reproducibility.

RHP-lipase were mixed in a 10-1 mass ratio and lyophilized to remove the aqueous solvent. A different ratio was used here compared to actual degradation studies because 80-1 RHP-lipase resulted in unstable droplets due to high RHP interfacial activity, preventing accurate measurement. PCL was dissolved first in toluene at a 0.5 mg/mL concentration. The PCL/toluene solution was then used to directly disperse RHP-lipase, giving a final concentration of 0.005 mg/mL for RHP and 0.0005 mg/mL for lipase in toluene. The concentration of each component was fixed across all groups. The water droplet was immersed in toluene after all three components (PCL, RHP, and lipase) were dispersed in toluene.

To determine whether PCL alone could disperse lipase in toluene, fluorescently labeled lipase was dissolved in the water phase (0.75 mg/mL concentration) while PCL was dissolved in the toluene phase (0.5 mg/mL). The fluorescence intensity of both phases did not change over a 3-hour period (data not shown), indicating the inability of PCL alone to disperse lipase in toluene via the water/toluene interface.

Section M8. Melt Processing, Thermal Treatment, and Operating Temperature to Program Degradation

PCL (10,000 g/mole) was first ground into a fine powder using a commercial grinder. RHP-lipase dried powder (1-1 mass ratio) was mixed with PCL powder and all three components were again passed through the commercial grinder. The PCL-RHP-lipase powder was then placed in a single-screw benchtop extruder, with a rotating speed of 20 RPM and an extrusion temperature of 85° C. Melt-extruded PCL-RHP-lipase filaments degrade with the same processive mechanism, as confirmed by GPC and LCMS.

For thermal treatment, PCL-RHP-lipase films were cast on microscope slides, placed on a hot plate at 80° C. for 5 mM to ensure complete melting, and crystallized at the specified temperature for up to 3 days to ensure complete recrystallization.

To determine the dependence of degradation on operating temperature, PCL-RHP-BC-lipase solution-cast films were placed in buffer at specified temperatures. For as-cast films, ramping temperature from 20° C. to ˜43° C. results in increased degradation rates. Further increases in temperature, however, result in degradation rate decreases. To rule out enzyme denaturation, the same small molecule assay described in section MS was employed at the given temperatures. Controls of just the 0.5 mM ester solution were run at each temperature to ensure that the ester was not self-hydrolyzing over the given measurement time period. The activity toward the small molecule significantly increases above 43° C., ruling out denaturation as the cause for reduced PCL degradation at high temperatures.

Section M9. RHPs with Different Compositions Enable PLA Depolymerization and Regulation of Embedded Enzyme Activity

RHPs' compositions were screened to determine the effects of RHP-enzyme interactions on depolymerization by embedded enzymes. Three compositions were chosen based on the segmental hydrophobicity, which was determined by simulating RHP sequences. Briefly, RHP sequences were generated using Compositional Drift.⁴³ The hydrophile-lipophile balance (HLB) value was used to evaluate the solubility of monomer side-chains through group contribution theory. Using the equation HLB=7+Σᵢ nᵢ HLBᵢ, where nᵢ is the number of the ith chemical group in the molecule with corresponding value HLBᵢ. The HLB value for each monomer side chain was estimated as: HLB(MMA)=8.45, HLB(EHMA)=5.12, HLB(OEGMA)=11.4 and HLB(SPMA)=18.5. A lower HLB value denotes higher hydrophobicity and a higher value means greater hydrophilicity. A Python program was created to continuously calculate the average segmental HLB values for a window sliding from the alpha to the omega ends of the simulated RHP chains. The window advanced by one monomer each time. We used a span containing odd numbers of monomers and assigned the average HLB value of that span to its middle monomer. Window size of 9 was used as an intermediate segmental region size. Hydropathy plots were generated to visualize randomly sampled sequences for each RHP composition and window size. An HLB-threshold=9 was set to distinguish hydrophobic and hydrophilic segments. The sequences are then averaged both across positions along the chain as well as across all 15,000 sequences in a simulated batch, to make batch-to-batch comparisons on the average segmental (window) hydrophobicity.

Similar tensiometry experiments as those outlined in section M7 were carried out using RHP (0.005 mg/mL)-proteinase K (0.0025 mg/mL), PLA and dichloromethane. PLA showed little interfacial activity. For the 20:50 MMA:EHMA RHP, addition of PLA measurably reduced the interfacial activity of the RHP. The 50:20 MMA:EHMA RHP had similar interfacial activity with or without PLA.

Section M10. Depolymerization in ASTM Composts or Tap Water

PCL-RHP-BC-lipase films were placed in tap water or an at-home compost setup. For water, films were submerged in 100 mL of tap water from a sink, and degradation proceeded identically over 24 hours (<95%) at the specified temperature. Soil was purchased from a local composting facility. The total dry organic weight of the soil was determined by leaving a known soil mass in an oven set to 110° C. overnight and then weighing the remaining material mass. Water was added to the soil to achieve a total moisture content of 50 or 60%, consistent with ASTM standards. For PCL-RHP-BC-lipase, up to 40% mass loss and 70% mass loss was observed after 2 and 4 days, respectively, in the compost setup at 40° C. For PLA-RHP-proteinase K, ˜34% mass loss occurred for 40 KDa PLA and ˜8% mass loss occurred for 85-160 KDa PLA after 5 days in a 50° C. soil compost.

Section S11. Oxidative Enzymes Embedded in Polyolefins

Manganese peroxidase from white rot fungus and laccase from Trametes versicolor were purchased from Sigma and used as purchased. RHP (50:20 MMA:EHMA) was mixed with either enzyme in a 4:1 ratio. Both enzymes were embedded in polyethylene (Mw=35 KDa) or polystyrene (Mn=260 KDa). For polyethylene, enzymes were embedded by solution casting from a 5 wt. % solution in toluene or melt pressing at 95° C. from polyethylene powder. For polystyrene, enzymes were embedded by resuspending directly in a 10 wt. % polystyrene in dichloromethane solution. Enzymes were embedded with and without mediators (Tween 80 for manganese peroxidase and hydroxybenzotriazole for laccase). The films were then placed in 30° C. or 60° C. malonate buffer (pH 4.5) for up to two weeks. After drying the films, infrared spectroscopy and GPC were used and no changes were observable for any enzyme-polyolefin system.

To confirm that enzymes were still active after embedding inside polyolefins, the films were submerged in a 1 mM solution of the small molecule 2,2′-Azino-bis(3-ethylbenzothiazoline-6-sulfonic acid) diammonium salt (ABTS) in malonate buffer. The solution turned dark blue for both manganese peroxidase and laccase, demonstrating that the embedded enzymes retained a high portion of activity. Tensiometry tests were carried out using RHP-manganese peroxidase or RHP-laccase with or with PS in toluene in the same setup and concentrations outlined for PCL/lipase. RHP-enzyme clusters with both enzymes achieved the same final interfacial tension with or without PS present and no lag phase or change in final interfacial tension, suggesting that the PS chains do not strongly interact with the enzymes.

Additional References Used in the Methods Section:
40 Bornscheuer, U. et al. Lipase of Pseudomonas-Cepacia for Biotechnological Purposes—Purification, Crystallization and Characterization. Bba-Gen Subjects 1201, 55-60, doi: 10.1016/0304-4165(94)90151-1 (1994).
41 Wurm, A. et al. Crystallization and Homogeneous Nucleation Kinetics of Poly(epsilon-caprolactone) (PCL) with Different Molar Masses. Macromolecules 45, 3816-3828, doi:10.1021/ma300363b (2012).
42 Ajioka, M., Suizu, H., Higuchi, C. & Kashima, T. Aliphatic polyesters and their copolymers synthesized through direct condensation polymerization. Polym Degrad Stabil 59, 137-143, doi: 10.1016/50141-3910(97)00165-1 (1998).
43 Smith, A. A. A., Hall, A., Wu, V. & Xu, T. Practical Prediction of Heteropolymer Composition and Drift. Acs Macro Lett 8, 36-40, doi:10.1021/acsmacrolett.8b00813 (2019).

Extended Data Table 1

| | | RHP | RHP MW | RHP | RHP:Enzyme | | Polymer:RHP:Enzyme |
|---|---|---|---|---|---|---|---|
| Sample | Enzyme | (MMA:EHMA:OEGMA:SPMA) | (kDa) | PDI | (mass ratio) | Polymer Host | (%) |
| p-BC1* | Purified BC- | 50:20:25:5 | 69 | 1.55 | 80:1 | PCL 80 kDa | 98.6:1.4:0.017 |
| | lipase | | | | | | |
| p-BC2^{†} | Purified BC- | 50:20:25:5 | 25 | 1.6 | 80:1 | PCL 80 kDa | 98.6:1.4:0.017 |
| | lipase | | | | | | |
| p-BC3 | Purified BC- | Tween 80 | N/A | N/A | 1:1^{‡} | PCL 80 kDa | 99.9:0.017:0.017 |
| | lipase | | | | | | |
| p-BC4 | Purified BC | 50:20:25:5 | 69 | 1.55 | 80:1 | PCL/PLLA | 98.6:1.4:0.017 |
| | lipase | | | | | Blend | |
| | | | | | | (75/25 and | |
| | | | | | | 50/50 by wt) | |
| p-BC5 | Purified BC | 50:20:25:5 | 69 | 1.55 | 80:1 | PCL-b-PLLA + | 98.6:1.4:0.017 |
| | lipase | | | | | pure PLLA | |
| | | | | | | (9/4 by wt) | |
| p-BC6 | Purified BC | 50:20:25:5 | 69 | 1.55 | 1:1 | PCL 10 kDa | 99.9:0.06:0.06 |
| | lipase | | | | | | |
| p-BC7 | Purified BC- | 60:10:25:5 | 18.5 | 1.45 | 80:1 | PCL 80 kDa | 98.6:1.4:0.017 |
| | lipase | | | | | | |
| p-BC8 | Purified BC- | 20:50:25:5 | 25.6 | 1.31 | 80:1 | PCL 80 kDa | 98.6:1.4:0.017 |
| | lipase | | | | | | |
| BC1 | BC-lipase | 50:20:25:5 | 69 | 1.55 | 2:1 | PCL 80 kDa | 94.9:3.4:1.7 |
| | blend | | | | | | |
| CA1 | CA-lipase | 50:20:25:5 | 69 | 1.55 | 2:1 | PCL 80 kDa | 94.9:3.4:1.7 |
| | blend | | | | | | |
| ProK1 | Proteinase K | 50:20:25:5 | 69 | 1.55 | 2:1 | PLLA 85-160 | 95.5:3.0:1.5 |
| | | | | | | kDa | |
| ProK2 | Proteinase K | 60:10:25:5 | 18.5 | 1.45 | 2:1 | PLLA 85-160 | 95.5:3.0:1.5 |
| | | | | | | kDa | |
| ProK3 | Proteinase K | 20:50:25:5 | 25.6 | 1.31 | 2:1 | PLLA 85-160 | 95.5:3.0:1.5 |
| | | | | | | kDa | |

*p-BC1 is the sample referred to as PCL-RHP-BC-lipase in the text.
**p-BC2 was tested, but no degradation data are provided in the text.
***Conditions simulate experiments from previous literature.

1. A method of programmable degradation of a plastic, the method comprising providing a plastic comprising a polymer, a nanoscopic dispersion of an enzyme comprising an site, and an enzyme protectant, wherein the active site and enzyme-protectant interactions are configured to provide processive depolymerization of the polymer as the primary degradation pathway with expanded substrate selectivity, and incubating the plastic under conditions to effect substantially complete depolymerization of the polymer without substantial microplastics formation with partial polymer degradation.

2. The method of claim 1, wherein: (a) the enzyme is lipase and the polymer/substrate is poly(caprolactone) (PCL), the lipase surface provides affinity to the polymer/substrate, and the active site has a relatively narrow deep entrance.

3. The method of claim 1, wherein: (b) the enzyme is proteinase K and the polymer/substrate is poly(lactic acid) (PLA) and the active site is relatively shallow and exposed.

4. The method of claim 1, wherein: (c) the protectant comprises random heteropolymers (RHPs) configured to nanoscopically disperse the enzymes and/or modulate activity or stability of the enzymes.

5. The method of claim 1, wherein: (d) the enzyme surface, protectant and polymer/substrate form complexes to sandwich the substrate between the enzyme and protectant.

6. The method of claim 1, wherein: (e) the polymer is a semi-crystalline polyester.

7. The method of claim 1, wherein: (f) the nanoscopic dispersion comprises 0.01 to 1.5 wt %.

8. The method of claim 1, wherein: (g) the enzyme is a hydrolase, such as a lipase or a proteinase.

9. The method of claim 1, wherein: (h) the depolymerization occurs in water or compost.

10. The method of claim 1, wherein: (i) the depolymerization occurs in 2 days in water, or in 30 days in compost.

11. The method of claim 1, wherein: (j) the depolymerization occurs at a temperature of 37-40 C.

12. The method of claim 1, wherein: (j) the depolymerization occurs at a temperature of 10-30 C, wherein depolymerization is facilitated by reducing crystalline lamellae thickness.

13. The method of claim 1, wherein: (k) the polymer is a polyester, and the enzyme comprises an active site matched with the polyester backbone.

14. The method of claim 1, wherein: (1) the plastic comprises lipase in poly(caprolactone) (PCL).

15. The method of claim 1, wherein: (m) the plastic comprises proteinase in poly(lactic acid) (PLA).

16. The method of claim 1, wherein: (n) the enzyme is a processive enzyme having a deep (1-4 nm), narrow (2-6 Å, at the base) hydrophobic cleft from its surface to the catalytic site to facilitate substrate polymer-chain sliding while preventing dissociation.

17. The method of claim 1, wherein: (o) the nanoscopically dispersed enzyme has a deep active site, and the polymer is a semi-crystalline polyesters degraded primarily via chain-end mediated processive depolymerization with programmable catalytic latency and material integrity.

18. The method of claim 1, wherein: (p) the enzyme has a surface-exposed active site, and the method realizes processive depolymerization (processivity) by engineering complexes of the enzyme, protectant and polymer.

19. The method of claim 1, wherein: (q) polycaprolactone and poly(lactic acid) containing less than 2 wt. % enzymes are depolymerized in days with up to 98% polymer-to-small molecule conversion in standard soil composts or household tap water, eliminating needs to separate and landfill their products in compost facilities,

20. A system for programmable degradation of a plastic, comprising a plastic comprising a polymer, a nanoscopic dispersion of an enzyme comprising an site, and an enzyme protectant, wherein the active site and enzyme-protectant interactions are configured to provide processive depolymerization of the polymer as the primary degradation pathway with expanded substrate selectivity to effect substantially complete depolymerization of the polymer without substantial microplastics formation with partial polymer degradation.
